SEQUENCE LISTING (3 sequences)

SEQ ID NO: 1 - protein, 10 aa, Artificial Sequence
  synthetic peptide
RTMPSWGKIF

SEQ ID NO: 2 - protein, 10 aa, Artificial Sequence
  synthetic peptide
MSDRKPESGS

SEQ ID NO: 3 - protein, 10 aa, Artificial Sequence
  synthetic peptide
TDFPGLLSYW

(19) United States
(12) Patent Application Publication
(10) Pub. No.: US 2006/0247424 A1
(43) Pub. Date: Nov. 2, 2006

(54) Anti-idiotype antibody, method of constructing the anti-idiotype antibody, and method of preparing idiotype antibody using the anti-idiotype antibody

(75) Inventor: Masatsugu Suzuki, Gunma (JP)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Peptide Door Co., Ltd, Gunma (JP)

(21) Appl. No.: 10/525,342

(22) PCT Filed: Aug. 22, 2003
(86) PCT No.: PCT/JP03/10671

§ 371 Date: Aug. 23, 2005

(30) Foreign Application Priority Data
Aug. 22, 2002 (JP) 2002-241695

(51) Int. Cl. C07K 16/42; C12P 21/04

(52) U.S. Cl. 530/387.2; 435/70.21

(57) ABSTRACT

The present invention provides: an anti-idiotype antibody which can be conveniently and economically produced compared with the existing type; a method of producing the anti-idiotype antibody; and a method of preparing a target idiotype antibody using the anti-idiotype antibody. A substance capable of binding to an antigen-binding site of a first antibody is prepared and this substance is ligated to a second antigen to give a fused antigen. Next, this fused antigen is bonded to a second antibody capable of binding to the second antigen, thereby giving an anti-idiotype antibody against the first antibody. In a method of preparing the idiotype antibody including: inoculating an animal with an anti-idiotype antibody against a specific idiotype antibody; and then further inoculating the animal with an antigen of the above idiotype antibody, the anti-idiotype antibody of the present invention is employed. Thus, the target idiotype antibody can be efficiently obtained.

### TECHNICAL FIELD

The present invention relates to an anti-idiotype antibody, which can be conveniently and economically produced, and a method of producing the same. The present invention further relates to a method of preparing an idiotype antibody using the anti-idiotype antibody.

### BACKGROUND ART

The antigen-binding sites of antibody molecules having different specificities for antigens differ in shape from each other and each antibody molecule has a unique antigen-binding site. Such an antigen-binding site has immunogenicity that is called an idiotype. In particular, an epitope having immunogenicity located on the periphery of this binding site is designated as an idiotope.

An antibody against the idiotope of an antibody is designated as an anti-idiotype antibody, which has been previously considered to control an immune reaction in a living body (e.g., Jerne N J, Ann Immunal (Paris) 1974; 125c: 373-378) and has been found in recent years to affect the expression of an antibody having the idiotope (idiotype antibody) (“Immunology Illustrated”, translated by Tomio Tada, Nankodo, issued on Feb. 10, 2000 pp. 117).

At present, in a method of producing the anti-idiotype antibody as usual method of producing typical antibodies, a variety of animals is inoculated and immunized with idiotype antibodies or idiotopes, and then the screening for anti-idiotype antibodies can be performed.

Such anti-idiotype antibodies have been utilized as various reagents and, in addition, increasingly developed in application to vaccines.

However, in conventional methods of producing anti-idiotype antibodies, there have been problems in that 1) the methods take time and costs as much as those for producing typical antibodies, 2) the screening for anti-idiotype antibodies is difficult, 3) animals must be sacrificed in every production, and so on.

### DISCLOSURE OF THE INVENTION

An object of the present invention is to provide: an anti-idiotype antibody which can be conveniently and economically produced in comparison with those produced by the existing method; a method of producing the same; and a method of preparing a target idiotype antibody in which the anti-idiotype antibody is utilized.

To achieve the above object, according to one aspect of the present invention, there is provided an anti-idiotype antibody which is capable of binding to a first antibody against a first antigen, characterized by comprising: a fused antigen including a substance which is capable of binding to an antigen-binding site of the first antibody and a second antigen, the substance being ligated to the second antigen; and a second antibody which is capable of binding to the second antigen.

For the anti-idiotype antibody of the present invention, it is preferred that the substance which is capable of binding to the antigen-binding site of the first antibody comprises an epitope of the first antigen.

In addition, the substance binding to the antigen-binding site of the first antibody is preferably a protein, a peptide, a carbohydrate, a lipid, a nucleic acid, or mixtures thereof.

Moreover, it is preferred that the substance which is capable of binding to the antigen-binding site of the first antibody is ligated to the second antigen via a spacer.

An anti-idiotype antibody of the present invention is composed of: a fused antigen in which a substance capable of binding to an antigen-binding site of a first antibody is ligated to a second antigen; and a second antibody capable of binding to the second antigen. Therefore, the substance capable of binding to the antigen-binding site of the first antibody can be simply altered depending on an idiotope of the first antibody, in order for the anti-idiotype antibody to function against the first antibody.

According to another aspect of the present invention, there is provided a method of producing an anti-idiotype antibody which is capable of binding to a first antibody against a first antigen, characterized by comprising: preparing a substance which is capable of binding to an antigen-binding site of the first antibody; ligating the substance to a second antigen to produce a fused antigen; and attaching the fused antigen to a second antibody which is capable of binding to the second antigen.

For the production method of the present invention, it is preferred that an epitope of the first antigen be prepared and this epitope be ligated to the second antigen.

It is also preferred that a protein, a peptide, a carbohydrate, a lipid, a nucleic acid, or complexes thereof which is capable of binding to the antigen-binding site of the first antibody be prepared and ligated to the second antigen.

Moreover, it is preferred that the substance which is capable of binding to the antigen-binding site of the first antibody be ligated to the second antigen via a spacer.

According to the production method of the present invention, a substance capable of binding to the antigen-binding site of the first antibody can be simply altered depending on an idiotope of the first antibody, thereby allowing the convenient and economical production of an anti-idiotype antibody against the first antibody.

According to still another aspect of the present invention, there is provided a method of preparing an idiotype antibody, comprising: inoculating an animal with an anti-idiotype antibody against a specific idiotype antibody; and further inoculating the animal with an antigen of the idiotype antibody to prepare the idiotype antibody, characterized by the improvement comprising an anti-idiotype antibody according to any one of claims 1 to 4 being used as the anti-idiotype antibody.

According to the preparation method of the present invention, an anti-idiotype antibody against a target idiotype antibody can be conveniently prepared. Therefore, the production of the idiotype antibody can be increased, thereby allowing the efficient preparation of the target idiotype antibody.

### BRIEF DESCRIPTION OF THE DRAWINGS

FIG. 1 is an explanatory view showing a method of producing an anti-idiotype antibody, in which an epitope of a first antigen, as it is, be used as a substance that is capable of binding to an antigen-binding site of a first antibody.

FIG. 2 is an explanatory view showing a method, in which a screening of a peptide that is capable of binding to an antigen-binding site of a first antibody is performed by a phage display method and the prepared peptide is used to produce an anti-idiotype antibody.

FIG. 3 is a graph showing a result of confirming by an ELISA method whether each of mouse anti-His-Tag antibodies (1) to (4) has become an anti-idiotype antibody against a rabbit anti-peptide A antibody.

FIG. 4 is a graph showing a result of confirming by an ELISA method whether each of mouse anti-biotin antibodies (5) to (8) has become an anti-idiotype antibody against a rabbit anti-peptide C antibody.

### BEST MODE FOR CARRYING OUT THE INVENTION

The anti-idiotype antibody of the present invention is an anti-idiotype antibody that is capable of binding to a first antibody against a first antigen, and is composed of: a fused antigen in which a substance capable of binding to an antigen-binding site of a first antibody is ligated to a second antigen; and a second antibody capable of binding to the second antigen.

In the present invention, a first antigen is not particularly limited as long as it is a substance available as an antigen. Specific examples of the first antigen include an antibody, a protein receptor, a hormone, an enzyme, a peptide, a nucleic acid, a glycoconjugate, a cell, a virus, and a compound of low-molecular weight.

A first antibody as an idiotype antibody may be prepared by a standard method using a given first antigen. Antibodies against a variety of antigens are commercially available and can be employed in the present invention.

Specific examples of a substance that is capable of binding to an antigen-binding site of a first antibody include a protein, a peptide, a carbohydrate, a lipid, a nucleic acid, and mixtures thereof. In the present invention, it is preferred that the substance be the smallest portion recognized and bonded by the antigen-binding site of the first antibody, that is, an epitope of the above-mentioned first antigen. Such an epitope of an antigen can be determined on the basis of information described in literatures and so on. For example, if the substance binding to the antigen-binding site of the first antibody is a peptide or a protein, the epitope can be also obtained by a phage display method (Smith, G. P., Science, 288, 1315-1317 (1985)). The phage display method is a method involving using a phage library allowed to display a foreign protein as a fused protein on the coat protein of a phage to screen a protein binding to a definite target substance. In the method, such a phage library is brought into being contact with a first antibody and a selection procedure (bio-panning) is carried out to thereby selectively obtain only phages expressing the foreign protein binding to the first antibody. By analysis of the phage DNA, the amino acid sequence of the foreign protein displayed on the surface of the phage can be easily identified. Then, based on this amino acid sequence, a peptide or a protein can be synthesized according to a method known in the art (such as a solid phase method or an Fmoc method) to easily prepare the desired peptide or protein in large amounts. The amino acid sequence and the number of amino acids of a peptide or a protein binding to the antigen-binding site of the first antibody is not necessarily defined because they vary depending on each first antibody. For example in the case of a peptide, it is usually preferred that the peptide is composed of 2-200 amino acids and is more preferred that a peptide is composed of 5-18 amino acids.

The phage library can be prepared, for example, by: chemically synthesizing randomized DNA; inserting the DNA into a gene encoding the coat protein of phage DNA; and introducing the DNA into E. coli according to those such as methods described by Smith, G. P., Science, 288, 1315-1317 (1985), and J. K. Scott and G. P. Smith, Science, 249, 386-390 (1990). However, any of those commercially available such as a trade name “Phage Display Peptide Library Kit” manufactured by New England Biolab can be also employed.

For an efficient screening of the peptide or protein binding to the antigen-binding site of the first antibody in the present invention, it is preferable to use a phage library having as many diversities as possible in a foreign protein displayed on the surface of the phage.

In the present invention, a second antigen is not particularly limited as long as it is a substance that can be chemically and physically ligated with the substance binding to the antigen-binding site of the first antibody, and specific examples of the second antigen include a peptide (e.g., His-Tag having plural histidines forming an amino bond), a carbohydrate, a nucleic acid, a lipid, and mixtures thereof, and a biotin. Preferably, the second antigen is any of those rendering an antibody thereagainst (i.e., a second antibody) easily obtainable.

The ligation between the substance capable of binding to the antigen-binding site of the first antibody and the second antigen varies in methods based on the kinds of the substance and the second antigen. However, the ligation may be carried out by a method known in the art according to each kind. For example, if peptides are used as the substance capable of binding to the antigen-binding site of the first antibody as well as the second antigen, the ligation method includes the following:

1) a peptide capable of binding to the antigen-binding site of the first antibody and a peptide being as the second antigen are synthesized as one consecutive peptide; and

2) a peptide being as an antigen, in which an amino acid residue having a functional group such as an amino, a thiol, or a carboxyl additively incorporated in the peptide sequence, is synthesized, and the functional group such as an amino, a thiol, or the like is activated, and then the peptide capable of binding to the antigen-binding site of the first antibody is ligated to the activated residue.

In the present invention, it is preferred that the ligation between the substance capable of binding to an antigen-binding site of a first antibody and the second antigen be carried out via a spacer. The spacer is not particularly limited as long as it is a substance through which the substance binding to the antigen-binding site of the first antibody can be chemically and physically ligated to the second antigen. More particularly, the spacer can be exemplified by a peptide, any of those each having a backbone with 2-18 carbon atoms (an ester bond or an ether bond may be contained in the backbone) and preferably having a hydrophilic functional group such as a hydroxyl group (e.g., polyvinyl alcohol having active groups at both ends, preferably polyvinyl alcohol having approximately 2-10 vinyl alcohol molecules polymerized), a sugar chain composed of 2-10 sugars, or the like. Preferable examples of the above-described peptide include a flexible linker such as a peptide in which plural glycines or serines forms peptide bond (usually 4-10 residues).

The ligation via such spacer can prevent the steric inhibition of binding between the peptide binding to the antigen-binding site of the first antibody and the first antibody. The length of the spacer may be selected as appropriate to be sufficient to avoid the steric inhibition of binding between the peptide binding to the antigen-binding site of the first antibody and the first antibody.

It is noted that a spacer may be previously ligated to the substance capable of binding to an antigen-binding site of a first antibody and subsequently to the second antigen, or may be previously ligated to the second antigen and subsequently to the substance capable of binding to an antigen-binding site of a first antibody. The ligation of the spacer to the substance binding to the antigen-binding site of the first antibody or the second antigen can be carried out by a method known in the art (see e.g., Motonori Ono, Yuichi Kanaoka, Humio Sakiyama, Hiroshi Maeda, “Biochemical Experimental Method 13, Chemical Modification of Proteins, vol. 2”, Japan Scientific Societies Press, pp. 81-113).

As a second antibody binding to the above-described second antigen, any of those easily available is preferred and a mouse antibody, a rabbit antibody, a human antibody, or the like can be selected for use in accordance with purposes and so on.

An anti-idiotype antibody of the present invention can be obtained by attaching a second antibody against the second antigen to a fused antigen, in which the substance capable of binding to an antigen-binding site of a first antibody is ligated to the second antigen as described above, through an antigen-antibody reaction.

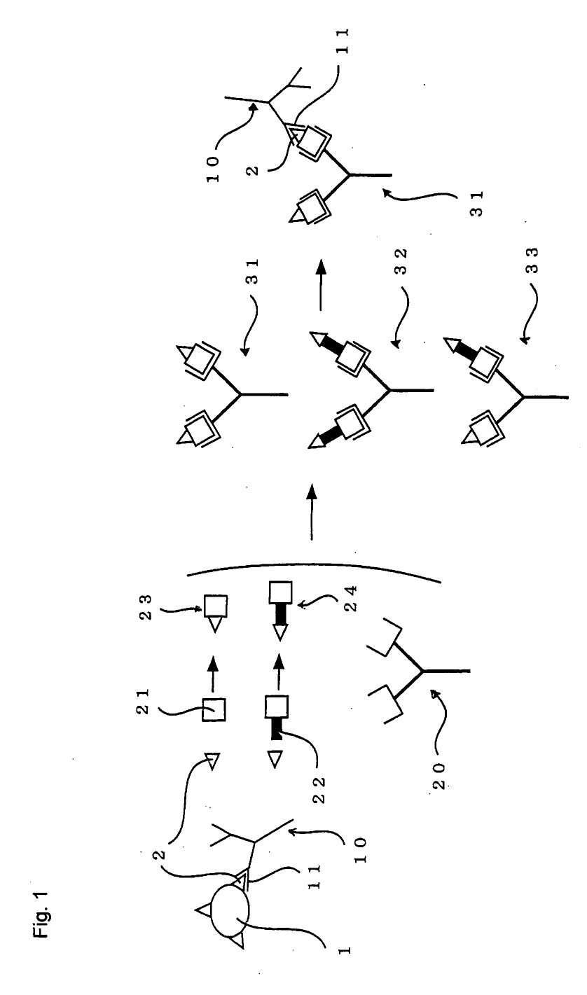

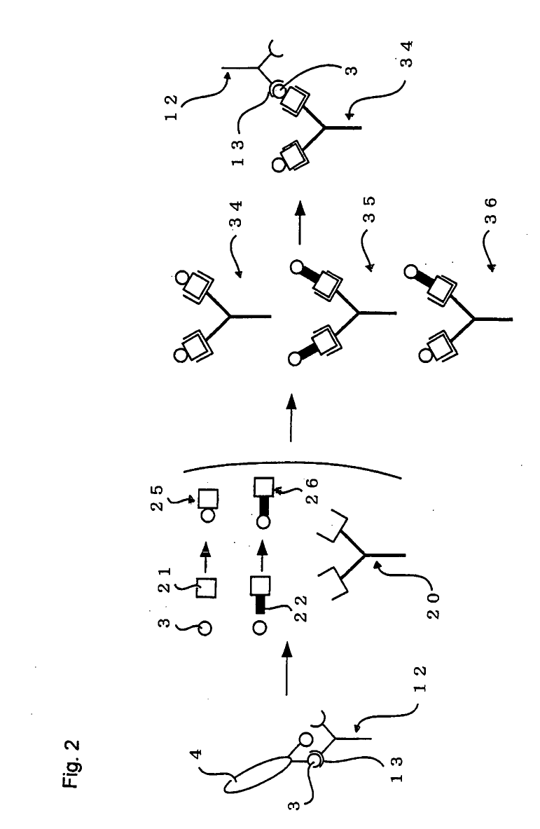

Hereinafter, a method of producing the anti-idiotype antibody of the present invention will be more fully described with reference to FIGS. 1 and 2.

FIG. 1 illustrates a method of producing an anti-idiotype antibody, in which an epitope of a first antigen, as it is, be used as a substance that is capable of binding to an antigen-binding site of a first antibody. This method is effective when the epitope of the first antigen has been already determined and the epitope can be prepared by a method known in the art.

Namely, an epitope 2 of a first antigen 1 is synthesized or prepared by a method known in the art on the basis of the result of analysis according to a standard method or on the basis of literature information and so on. Thereafter, this epitope 2 is directly ligated to a second antigen 21 to thereby produce a fused antigen 23. This fused antigen 23 can be then attached to a second antibody 20 against the second antigen 21 through an antigen-antibody reaction to thereby produce an anti-idiotype antibody 31 against a first antibody 10. In addition, by using a fused antigen 24 in which the epitope 2 is ligated to the second antigen 21 via a spacer 22, an anti-idiotype antibody 32 can be produced. By using both of the fused antigens 23 and 24, an anti-idiotype antibody 33 can be produced.

Each of the anti-idiotype antibodies 31 to 33 obtained as above can be bonded to an antigen-binding site 11 of the first antibody 10 via the epitope 2, as shown in the figure.

FIG. 2 illustrates a method, in which a screening of the substance that is capable of binding to an antigen-binding site of a first antibody is performed, that is, an epitope of the first antibody is analyzed by a phage display method, and the prepared peptide capable of binding to the antigen-binding site of the first antibody on the basis of the result of the analysis is used to produce an anti-idiotype antibody. This method is effective when the first antibody (idiotype antibody) has been already obtained. Incidentally, in the description below, the same reference numerals will be used to designate the substantially same components as those in the above description, so the description thereof will be omitted.

That is, a phage 4 displaying a peptide 3 capable of binding to an antigen-binding site 13 of a first antibody 12 is obtained in a screening by a phage display method, and its amino acid sequence is analyzed to synthesize the peptide 3 by a method known in the art. Subsequently, the peptide 3 is directly ligated to a second antigen 21 to thereby produce a fused antigen 25. This fused antigen 25 can be then attached to a second antibody 20 against the second antigen 21 through an antigen-antibody reaction to thereby produce an anti-idiotype antibody 34. In addition, by using a fused antigen 26 in which the peptide 3 is ligated to the second antigen 21 via a spacer 22, an anti-idiotype antibody 35 can be produced. By using both of the fused antigens 25 and 26, an anti-idiotype antibody 36 can be produced.

Each of the anti-idiotype antibodies 34 to 36 obtained as above can be bonded to an antigen-binding site 13 of the first antibody 12 via the peptide 3, as shown in the figure.

The anti-idiotype antibody of the present invention may be used in various applications as a substitute for conventional anti-idiotype antibodies, for example in a variety of detection/measurement reagents and pharmaceuticals such as vaccines.

As described in e.g., “Immunology Illustrated” (translated by Tomio Tada, Nankodo, issued on Feb. 10, 2000, pp. 117), it has been known that the production of an idiotype antibody is dramatically increased by: inoculating an animal with an appropriate amount of an anti-idiotype antibody against a specific idiotype antibody; and then further inoculating the animal with an antigen (first antigen) of the idiotype antibody. Thus, in the method of preparing an idiotype antibody as mentioned above, the use of the anti-idiotype antibody of the present invention allows the efficient preparation of a target idiotype antibody. Examples of the above-described animal include a mouse, a rat, a rabbit, a goat, a horse, a cow, a pig, a chicken, and a human, which has been generally used for the production of antibodies.

### EXAMPLES

Although the present invention will be more particularly described hereinafter with reference to examples, the present invention is not intended to be limited to them.

### Example 1

An arbitrary peptide, more particularly a peptide having a sequence shown in SEQ ID NO.: 1 (hereinafter, referred to as a peptide A) was used as a first antigen and a rabbit was immunized with the peptide according to a general method of producing an anti-peptide antibody. After three times of test blood-collection, an anti-peptide A antibody was gained in a sufficient amount. Therefore, after collection of the blood, a rabbit anti-peptide A antibody (first antibody) in the collected blood was purified with a column on which the peptide A was immobilized.

A peptide (1), in which His-Tag (a second antigen; a peptide having four histidines bonded) was fused to the C-terminus of the peptide A, and a peptide (2), in which two of serines were inserted as a spacer to the C-terminus of the peptide A and His-Tag was consecutively followed thereto, were respectively synthesized according to a standard method. Likewise, a peptide (3), in which His-Tag was fused to the C-terminus of a peptide having a sequence shown in SEQ ID NO.: 2 (hereinafter, referred to as a peptide B), and a peptide (4), in which two of serines were inserted as a spacer to the C-terminus of the peptide B and His-Tag was consecutively followed thereto, were respectively synthesized as controls.

Each of the above peptides (1) to (4) was attached to a mouse antibody capable of binding to His-Tag (a second antibody; trade name “Anti-Histag Antibody” manufactured by CN Bioscience Inc.) through an antigen-antibody reaction. Hereinafter, the mouse anti-His-Tag antibody bonded with the peptide (1) is referred to as a mouse anti-His-Tag antibody (1), the mouse anti-His-Tag antibody bonded with the peptide (2) is referred to as a mouse anti-His-Tag antibody (2), the mouse anti-His-Tag antibody bonded with the peptide (3) is referred to as a mouse anti-His-Tag antibody (3), and the mouse anti-His-Tag antibody bonded with the peptide (4) is referred to as a mouse anti-His-Tag antibody (4).

Thereafter, it was confirmed by an ELISA method whether each of the mouse anti-His-Tag antibodies (1) to (4) is being as an anti-idiotype antibody against the rabbit anti-peptide A antibody. More particularly, the rabbit anti-peptide A antibody was dissolved in a 100 mM sodium carbonate buffer (pH 8.0) to be a 10-μg/mL solution. One hundred micro litter of the solution was placed per well of a 96 -well plate manufactured by Corning (high binding type), followed by letting the plate stand at 25° C. for 1 hour to carry out immobilization by physical absorption. After having been washed, the plate was blocked with 250 μL/well of a 2% (w/v) skimmilk solution (100 mM sodium carbonate buffer, pH 8.0). On the other hand, without the immobilization of the rabbit anti-peptide A antibody, wells blocked with 250 μL of a 2% (w/v) skim milk solution (100 mM sodium carbonate buffer, pH 8.0) were prepared as the controls.

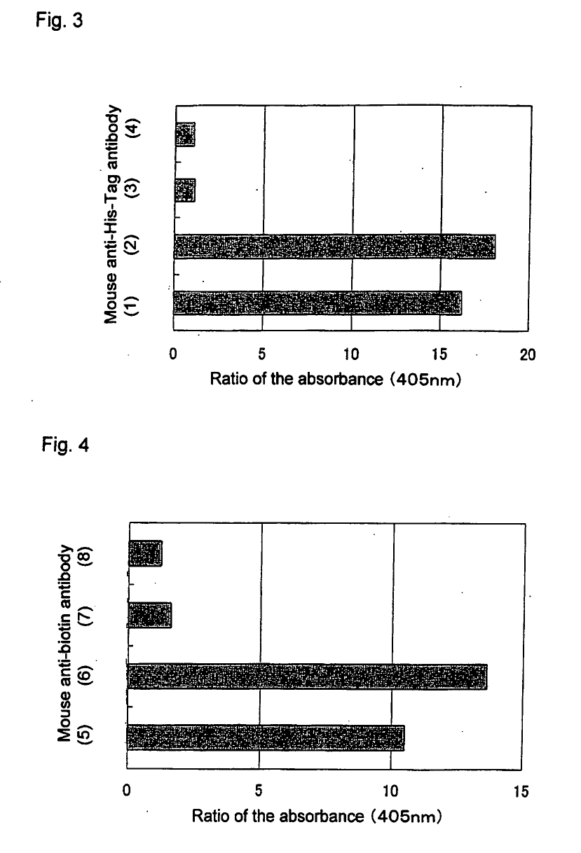

The mouse anti-His-Tag antibodies (1) to (4) each were dissolved in a 2% (w/v) skim milk solution (100 mM sodium carbonate buffer, pH 8.0) to be a 1-μg/mL solution. Two hundred micro litter of the solution was placed into three of the rabbit anti-peptide A antibody-immobilized wells and into two of wells that were only blocked, followed by letting the wells stand at 25° C for 1 hour. After the plate had been washed, each of the wells was supplemented with an anti-mouse antibody labeled with HRP and left to stand at 25° C. for 1 hour. After the plate had been washed, an ABTS reaction solution was added thereto. Subsequently, the level of binding of the fused antibodies was measured at an absorbance of 405 nm by determining the ratio of the absorbance (the mean value of the absorbances from three of the rabbit anti-peptide A antibody-immobilized wells/the mean value of the absorbances from two of wells that were only blocked). The result is shown in FIG. 3.

As shown in FIG. 3, the binding to the rabbit anti-peptide A antibody was observed with the mouse anti-His-Tag antibodies (1) and (2), where these antibodies are being as anti-idiotype antibodies against the rabbit anti-peptide A antibody. Especially, the mouse anti-His-Tag antibody (2), into which the spacer was inserted, was shown to bind to the rabbit anti-peptide A antibody more readily than the mouse anti-His-Tag antibody (1) carrying no spacer.

### Example 2

An arbitrary peptide, more particularly a peptide having a sequence shown in SEQ ID NO.: 3 (hereinafter, referred to as a peptide C) was used as a first antigen and an antibody capable binding to the peptide C (a first antibody; hereinafter, referred to as a rabbit anti-peptide C antibody) was purified in the same manner as in Example 1.

A peptide (5), in which a biotin (a second antigen) was directly linked to the N-terminus of the peptide C, and a peptide (6), in which a biotin was linked to the N-terminus of the peptide C via a spacer using trade name “sulfo NHS-LC Biotin” (manufactured by Pierce), were prepared. Likewise, a peptide (7), in which a biotin was directly linked to the N-terminus of the above peptide B, and a peptide (8), in which a biotin was linked to the N-terminus of the peptide B via a spacer using trade name “sulfo NHS-LC Biotin” (manufactured by Pierce), were prepared as controls.

Each of the above peptides (5) to (8) was attached to a mouse antibody capable binding to biotin (a second antibody; trade name “anti Biotin antibody” manufactured by Dianova GmbH) through an antigen-antibody reaction. Hereinafter, the mouse anti-biotin antibody bonded with the peptide (5) is referred to as a mouse anti-biotin antibody (5), the mouse anti-biotin antibody bonded with the peptide (6) is referred to as a mouse anti-biotin antibody (6), the mouse anti-biotin antibody bonded with the peptide (7) is referred to as a mouse anti-biotin antibody (7), and the mouse anti-biotin antibody bonded with the peptide (8) is referred to as a mouse anti-biotin antibody (8) in the same manner as in Example 1.

Thereafter, it was confirmed by an ELISA method whether each of the mouse anti-biotin antibodies (5) to (8) is being as an anti-idiotype antibody against the rabbit anti-peptide C antibody in the same manner as in Example 1. The result is shown in FIG. 4.

As shown in FIG. 4, the binding to the rabbit anti-peptide C antibody was observed with the mouse anti-biotin antibodies (5) and (6), where these antibodies are being as anti-idiotype antibodies against the rabbit anti-peptide C antibody. Especially, the mouse anti-biotin antibody (6), into which the spacer was inserted, was shown to bind to the rabbit anti-peptide C antibody more readily than the mouse anti-biotin antibody (5) carrying no spacer. In this case, it is suggested that, because of the use of a small substance such as biotin as a second antigen, the effect of the spacer gets more noticeable.

(Sequence Listing Free Text)

SEQ ID NO.: 1: a peptide used as a first antigen.
SEQ ID NO.: 2: a peptide incapable of binding to an antigen-binding site of a first antibody.
SEQ ID NO.: 3: a peptide used as a first antigen.

### INDUSTRIAL APPLICABILITY

As described above, according to the present invention, there is provided an anti-idiotype antibody against a first antibody can be conveniently and economically produced by: preparing a substance capable of binding to an antigen-binding site of the first antibody; ligating the substance to a second antigen to produce a fused antigen; and attaching the fused antigen to a second antibody binding to the second antigen.

The anti-idiotype antibody of the present invention can be utilized in, for example, a variety of detection/measurement reagents and pharmaceuticals such as vaccines. In addition, the use of the anti-idiotype antibody of the present invention allows the efficient preparation of a target idiotype antibody, as described above.

1. An anti-idiotype antibody which is capable of binding to a first antibody against a first antigen, characterized by comprising: a fused antigen including a substance which is capable of binding to an antigen-binding site of the first antibody and a second antigen, the substance being ligated to the second antigen; and a second antibody which is capable of binding to the second antigen.

2. An anti-idiotype antibody according to claim 1, wherein the substance which is capable of binding to the antigen-binding site of the first antibody comprises an epitope of the first antigen.

3. An anti-idiotype antibody according to claim 1 or 2, wherein the substance which is capable of binding to the antigen-binding site of the first antibody comprises a protein, a peptide, a carbohydrate, a lipid, a nucleic acid, or mixtures thereof.

4. An anti-idiotype antibody according to any one of claims 1 to 3, wherein the substance which is capable of binding to the antigen-binding site of the first antibody is ligated to the second antigen via a spacer.

5. A method of producing an anti-idiotype antibody which is capable of binding to a first antibody against a first antigen, characterized by comprising: preparing a substance which is capable of binding to an antigen-binding site of the first antibody; ligating the substance to a second antigen to produce a fused antigen; and attaching the fused antigen to a second antibody which is capable of binding to the second antigen.

6. A method of producing an anti-idiotype antibody according to claim 5, wherein an epitope of the first antigen is prepared and the epitope is ligated to the second antigen.

7. A method of producing an anti-idiotype antibody according to claim 5 or 6, wherein a protein, a peptide, a carbohydrate, a lipid, a nucleic acid, or mixtures thereof which is capable of binding to the antigen-binding site of the first antibody is prepared and ligated to the second antigen.

8. A method of producing an anti-idiotype antibody according to any one of claims 5 to 7, wherein the substance which is capable of binding to the antigen-binding site of the first antibody is ligated to the second antigen via a spacer.

9. In a method of preparing an idiotype antibody, comprising: inoculating an animal with an anti-idiotype antibody against a specific idiotype antibody; and further inoculating the animal with an antigen of the idiotype antibody to prepare the idiotype antibody; the improvement comprising an anti-idiotype antibody according to any one of claims 1 to 4 being used as the anti-idiotype antibody.
